SEQUENCE LISTING (4 sequences)

SEQ ID NO: 1 - DNA, 339 bp, Mice
caggtgcagctgaaggagtcaggacctggcctggtggcgccctcacagagcctgtccatcacttgcactgtctctgggctttcattaaccagctatggtgtacactgggttcgtcaggccccaggaaagggtctggagtggctgggagtaatttggggtggtggaaacacaaattataattcggctctcatgtccagactgagcatcagcaaagacaactccaggagccaagttttcttaagaatgaacagtctgcaaattgatgacacagccatgtactattgtgccagaggcaggatggactactggggtcaaggaacctcagtcaccgtctcgtca

SEQ ID NO: 2 - DNA, 315 bp, Mice
gacatcaagatgacccagtctccatcttccatgtatgcatctctaggagaaagagtcactatcacttgcaaggcgagtcaggacattagtagctatttaggctggttacagcagaaaccagggaaatctcctaagaccctgatctatcgtgcaaacacattggtagaaggggtcccatccagattcagtggcagtggatctggggaagattattctctcaccatcagcagcctggagtatgaagatatgggaatttattattgtctacagtatgatgagtttccgtacacgttcggaggggggaccaagctggaa

SEQ ID NO: 3 - protein, 113 aa, Mice
QVQLKESGPGLVAPSQSLSITCTVSGLSLTSYGVHWVRQAPGKELEWLGVIWGGGNTNYNSALMSRLSISKDNSRSQVFLRMNSLQIDDTAMYYCARGRMDYWGQGTSVTVSS

SEQ ID NO: 4 - protein, 105 aa, Mice
DIKMTQSPSSMYASLGERVTITCKASQDISSYLGTLQQKPGKSPKTLIYRANTLVEGVPSRFSGSGSGEDYSLTISSLETEDMGIYYCLQYDEFPYTFGGGTKLE

(19) United States
(12) Patent Application Publication
(10) Pub. No.: US 2018/0209966 A1
(43) Pub. Date: Jul. 26, 2018

(54) FLUORESCENCE POLARIZATION IMMUNOASSAY METHOD FOR DETECTING CARBARYL

(71) Applicant: OILCROPS RESEARCH INSTITUTE OF CHINESE ACADAMY OF AGRICULTURE SCIENCES, HUBEI (CN)

(72) Inventors: Peiwu LI, Hubei (CN); Hui LI, Hubei (CN); Qingqing YANG, Hubei (CN); Xiaoqian TANG, Hubei (CN); Qi ZHANG, Hubei (CN); Wen ZHANG, Hubei (CN); Zhaowei ZHANG, Hubei (CN); Xiaoxia DING, Hubei (CN)

(73) Assignee: OILCROPS RESEARCH INSTITUTE OF CHINESE ACADAMY OF AGRICULTURE SCIENCES, HUBEI (CN)

(21) Appl. No.: 15/879,440

(22) Filed: Jan. 25, 2018

(30) Foreign Application Priority Data
Jan. 25, 2017 (CN) 201710061067.X

(51) Int. Cl. G01N 33/533

(52) U.S. Cl. CPC G01N 33/533

(57) ABSTRACT

A fluorescence polarization immunoassay (FPIA) method for detecting carbaryl. The method includes the steps of mixing a sample to be tested, a fluorescent marker solution and an anti-carbaryl monoclonal antibody solution; incubating for carrying out a competitive reaction; determining a fluorescence polarization value of the resulting system; calculating the concentration of carbaryl in the sample to be tested according to a standard curve of fluorescence polarization values-carbaryl concentrations in carbaryl standard samples. According to the FPIA method for detecting carbaryl provided in the present invention, only the addition of a sample is required, and no separation and washing operations are needed.

### CROSS-REFERENCE TO RELATED APPLICATION

This application claims the priority benefit of China application serial no. 201710061067.X, filed on Jan. 25, 2017. The entirety of the above-mentioned patent application is hereby incorporated by reference herein and made a part of this specification.

### BACKGROUND OF THE INVENTION

### 1. Field of the Invention

The present invention relates to the field of immunoassay technologies and pesticide residue detection, and in particular to a fluorescence polarization immunoassay (FPIA) method for detecting carbaryl.

### 2. Description of Related Art

Carbaryl, also known as Sevin, is a carbamate insecticide having characteristics such as high potency, long duration of action and high selectivity. Carbaryl is a broad-spectrum insecticide widely used on fruits, vegetables, cotton, tea and other crops in China. Carbaryl is a kind of medium toxic pesticide, which has contact and stomach poisoning functions and easily causes the issues of excessive residue in agricultural products and safe consumption because of its long-persist in water, soil, fruits, food and so on, thus bringing damage to the immune system, nerve centre and endocrine system of human, and threatening the sustainable development of agricultural industry. In 2008, the Annex II of the fourth meeting of the EU Chemical Review Committee clearly put forward the ban on the use of carbaryl, and it is considered that carbaryl can induce carcinogenic effects on all organs in human (where gastrointestinal cancer is most common) in addition to the ingestion and inhalation poisoning, and belongs to Group 3 carcinogens. GB2763-2014 National food safety standard-Maximum residue limits for pesticides in food prescribes that the maximum residue limit (MRL) of carbaryl is 1 ppm in vegetables, 0.5-1 ppm in cereals and 1 ppm in tea. The MRL of carbaryl in fruits is not specified in the standard, and is 1-5 ppm with reference to the provisions in other countries.

Currently, the methods for detecting carbaryl residue mainly include high performance liquid chromatography (HPLC), gas chromatography (GC)-mass spectrometry (MS), HPLC-MS, cholinesterase inhibition, and immunoassay. Among them, although HPLC, GC-MS and HPLC-MS have the advantages of high accuracy and good reproducibility, they usually need expensive equipment, skilled operators and stringent experimental conditions. Therefore, it is difficult to meet the requirements of rapid detection on site. Cholinesterase inhibition method which has the advantage of strong versatility, simple and fast, can meet the need of on-site detection to some extent. However, due to the low sensitivity, it is difficult to realize trace detection.

Immunoassay technology is widely used in the field of pesticide residue detection because of its advantages of high sensitivity, high specificity, low cost, and being simple and rapid. However, multiple steps of separation and washing operations are needed in the commonly used enzyme-linked immunosorbent assay (ELISA) and lateral flow immunochromatographic assay, so the operation is complex and time-consuming.

### SUMMARY OF THE INVENTION

In view of the disadvantages of complex operation and waste of time existing in the immunoassay technology for detecting carbaryl in the prior art, a fluorescence polarization immunoassay (FPIA) method for detecting carbaryl is established, in which the entire detection process requires only one step of competitive reaction, so the operation is simple and fast.

The FPIA method for detecting carbaryl provided in the present invention comprises the steps of mixing a sample to be tested, a fluorescent marker solution and an anti-carbaryl monoclonal antibody solution; incubating for competitive reaction;

determining a fluorescence polarization value of the resulting system; and calculating the concentration of carbaryl in the sample to be tested according to a standard curve between the known concentrations of carbaryl and the corresponding fluorescence polarization values.

In accordance with the present invention, the standard curve between the known concentrations of carbaryl and the corresponding fluorescence polarization values is obtained by mixing a series of given concentrations of carbaryl standard solutions respectively with the fluorescent marker solution and the anti-carbaryl monoclonal antibody solution, incubating for competitive reaction, and determining the fluorescence polarization values of the resulting systems; and plotting a standard curve of the determined fluorescence polarization values as longitudinal coordinates against the concentrations of the series of given concentrations of carbaryl standard solutions as horizontal coordinates.

In accordance with the present invention the fluorescent marker is a conjugate of a carbaryl hapten 6-(1-naphthyloxyformamido)-hexanoic acid (CNH) with fluorescein via an amide linkage.

In accordance with the present invention, the fluorescein is any one selected from fluorescein isothiocyanate ethylenediamine (EDF), fluorescein isothiocyanate butanediamine (BDF), and fluorescein isothiocyanate hexamethylenediamine (HDF), and the fluorescent marker has a structure as shown in Table 1:

TABLE 1

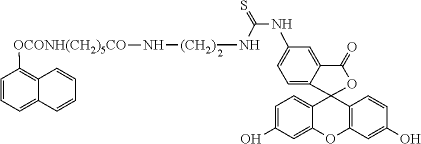

CNH-EDF

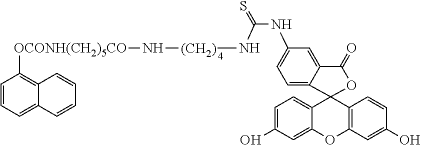

CNH-BDF
or

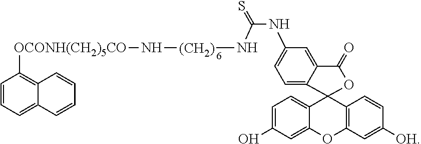

CNH-HDF

In accordance with the present invention, the fluorescent marker is preferably CNH-EDF.

In accordance with the present invention, the anti-carbaryl monoclonal antibody is secreted by the hybridoma cell line Jnw1D2 deposited in China Center for Type Culture Collection (CCTCC), Wuhan University, Wuhan, China on Mar. 29, 2016 with the accession number of CCTCC No. C201654.

In accordance with the present invention, the concentrations of the series of given concentrations of carbaryl standard solutions are 9 concentration gradients including 100 μg/mL, 10 μg/mL, 1μg/mL, 0.5 μg/mL, 0.1 μg/mL, 0.05 μg/mL, 0.01 μg/mL, 0.005 μg/mL, and 0.001 μg/mL.

In accordance with present invention, the solvent for formulating the fluorescent marker solution is a borate buffer solution; and the working concentration of the fluorescent marker is a corresponding concentration at which the fluorescence intensity of the fluorescent marker is 10 times of the background value of the borate buffer solution, and is 5 nM.

In accordance with present invention, the working concentration of the anti-carbaryl monoclonal antibody is a corresponding antibody dilution factor when 70% of the anti-carbaryl monoclonal antibody binds to the fluorescent marker, and is 1 μg/mL.

In accordance with present invention, the volume of the sample solution to be tested is 50 μL, and the volume of the fluorescent marker solution and the anti-carbaryl monoclonal antibody solution is respectively 500 μL.

In accordance with the present invention, the competitive reaction occurs at 20-25° C. and preferably 25° C., and is continued for 5-10 min, and preferably 10 min.

In accordance with the present invention, the fluorescence polarization value is determined at an excitation wavelength of 485 nm and an emission wavelength of 530 nm.

In accordance with the present invention, the sample to be tested is an agricultural product such as fruits and vegetables, and specifically strawberry.

The pretreatment procedures for strawberry samples before detection are as follows: the strawberry sample was mashed and homogenized with acetonitrile, then filtered into a centrifuge tube containing sodium chloride to collect the filtrate. After oscillating for a while, the sample was centrifuged to obtain the supernatant extract. Then the solvent of the supernatant was evaporated. The sample residue was dissolved in methanol to obtain the sample matrix solution.

When the sample to be tested is strawberry, a series of given concentrations of carbaryl standard solutions are formulated with the sample matrix solution obtained by pre-treating a blank strawberry sample, and amenable to FPIA assay, to obtain a standard curve of the strawberry matrix.

The present invention further provides use of the method in the detection of carbaryl content in agricultural products.

The detection principle in the present invention is that the anti-carbaryl monoclonal antibody specifically binds to the fluorescein-labelled carbaryl hapten, such that the fluorescence polarization signal intensity of the fluorescein is increased. If carbaryl is present in a sample to be tested (or in a carbaryl standard), carbaryl competes for binding to the anti-carbaryl monoclonal antibody with the fluorescein-labelled carbaryl hapten, such that the number of the fluorescein-labelled carbaryl hapten bound to the anti-carbaryl monoclonal antibody is reduced, which results in the decline of the fluorescence polarization signal intensity with increasing concentrations of carbaryl. Therefore, fast, sensitive and quantitative detection of carbaryl can be achieved in the present invention by using the highly sensitive and specific anti-carbaryl monoclonal antibody.

The beneficial effects of the present invention are as follows.

The existing carbaryl detection technology is cumbersome and time consuming and cannot meet the requirement of high-throughput rapid detection. Most of other immunoassay methods involve a heterogeneous reaction and require multiple incubation and washing steps, which are thus time consuming and complex in operation. In view of these defects, the present invention provides a homogeneous and fast fluorescence polarization immunoassay (FPIA) method that requires only addition of a sample and no separation and washing operations, so that a detection result can be obtained within ten minutes. In the FPIA method for detecting carbaryl established in the present invention, the sensitivity of the standard curve in the borate buffer solution is 82.3 ng/mL, and the detection range is 17.7-383.4 ng/mL; and the detection sensitivity in the sample is 108.6 μg/kg and the detection range is 32.4-363.6 μg/kg. The method is rapid, simple and high-throughput, can meet the residue detection limit of carbaryl, and is extremely suitable for the detection of carbaryl in strawberry samples.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 shows a standard curve for detecting carbaryl by FPIA in a borate buffer solution.

FIG. 2 shows a standard curve for detecting carbaryl by FPIA in a strawberry sample matrix.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

The present invention will be described below by way of specific examples, but the present invention is not limited thereto.

Unless otherwise specified, the experimental methods used in the following examples are conventional methods. Reagents, biological materials and the like used in the following examples are commercially available unless otherwise specified.

The anti-carbaryl monoclonal antibody used in the following examples is a monoclonal antibody secreted by the hybridoma cell line Jnw1D2, which is specifically obtained through a process below.

Screening of Hybridoma Cell Line Jnw1D2

1. Immunization of Animals

6 6-week old BALB/c mice were immunized with carbaryl immunogen CNH-BSA obtained by conjugating 6-(1-naphthyloxyformamido)-hexanoic acid (CNH) to bovine serum albumin (BSA). During the first immunization, the carbaryl immunogen was emulsified with an equal volume of Freund's complete adjuvant, and subcutaneously injected to the mice at multiple sites in the cervicodorsal region. The second immunization was carried out 3 weeks later, in which the carbaryl immunogen was emulsified with an equal volume of Freund's incomplete adjuvant, and subcutaneously injected to the mice at multiple sites in the cervicodorsal region. The third and fourth immunizations were carried out with an interval of two weeks from the previous immunization, and the immunization process was the same as the second immunization. The same dose of 100 μg/animal was used in the four immunizations. On the 7th day after the third immunization, blood was collected from the tail vein of mice and the serum was separated. The serum titer in the mice was monitored by indirect ELISA and the serum sensitivity in the mice was measured by indirect competitive ELISA. Mice corresponding to the serum with high titer and high sensitivity were boosted with a 2-fold immunization dose.

2. Cell Fusion

3 days after boost immunization, cell fusion was carried out by a conventional method using 50% by weight of polyethylene glycol, i.e., PEG (molecular weight: 1450) as a fusion agent. Specifically, the steps were as follows. Mice to be fused were sacrificed by cervical dislocation under aseptic conditions. The spleen cells were isolated, mixed with murine myeloma cells SP2/0 at a ratio of 5:1, washed with RPMI-1640 basal medium, and centrifuged for 5 min at 1200 rpm. The supernatant was discarded and the residue was drained. 1 mL of PEG was added and fused for 1 minute. RPMI-1640 basal medium was slowly added and centrifuged. The supernatant was discarded, and the pellet was the fused cells, which were re-suspend in 20 mL HAT complete medium. The suspended cells were added to 80 mL semi-solid medium, mixed, added to a 6-well cell culture plate in an amount of 2 mL/well, and then incubated in a CO₂ incubator at 37° C.

The complete cell culture medium containing 1% HAT contained 20% by volume (vol %) of fetal calf serum, 75 vol % of RPMI-1640 basal medium, 1% by weight (wt %) of L-glutamine, 1 vol % of HEPES, 1 vol % of double antibody (10,000 units per milliliter of penicillin and 10,000 micrograms per milliliter of streptomycin), 2 vol % of a growth factor (HFCS) and 1 wt % of hypoxanthine-aminopterin-thymidine (HAT) and methyl cellulose, which were purchased from sigma-Aldrich.

3. Screening and Cloning of Cell Lines

2-3 weeks after cell fusion, when the cell colonies grew up to be visually visible, the clones were picked out from the culture medium with a micropipette, transferred to a 96-well cell culture plate and cultured in HAT liquid. When the cells grew to cover ⅔ of the bottom of the well, the culture supernatant was aspirated for testing. A two-step screening method was adopted. In the first step, indirect ELISA method was used to screen out the positive wells against carbaryl, but not the carrier protein BSA. In the second step, indirect competitive ELISA method was used for testing the positive wells screened out in the first step with carbaryl as a competitor. Wells with high absorbance and sensitivity were chosen (where the high absorbance means a high final value determined in the well where the competitor is 0, i.e., the positive control well, and the high sensitivity means a low concentration, i.e. the IC50, of the competitor achieving 50% inhibition). The cells were subcloned by limiting dilution analysis, and then detected by the two-step method as described above. After 4-5 rounds of repeated subcloning, a hybridoma cell line Jnw1D2 was obtained, which was deposited in China Center for Type Culture Collection (CCTCC) (Wuhan University, Wuhan, China) under the CCTCC Accession No: C201654.

4. Sequencing of antibody variable region of anti-carbaryl monoclonal antibody in hybridoma cell line Jnw1D2

(1) Total RNA extraction: The total RNA was extracted from the hybridoma cell line Jnw1D2 by using the total RNA extraction kit of Tiangen Company following the instructions.

(2) cDNA synthesis: Using the total RNA obtained in Step 1 as a template and oligo(dT)15 as a primer, a first strand cDNA was synthesized by reverse transcription following the instructions of SuperScript™-2II reverse transcriptase, where the primer oligo(dT)15 was purchased from Invitrogen.

(3) Cloning of the variable region gene by PCR: According to the conserved sites in the mouse antibody gene sequence in GENEBANK, primers were designed, and the light and heavy chain variable region genes of the antibody were amplified by using the cDNA as a template. PCR program: 30 cycles of 30 sat 94° C., 45 sat 58° C., and 1 min at 72° C., following by extension at 72° C. for 10 min. After the PCR product was separated by 1% (wt) agarose gel electrophoresis, the DNA fragment was purified and recovered with a kit, ligated to the vector pMD18-T, and transformed into E. coli DH5α competent cells. The positive clones were picked up and sequenced by Shanghai Sunny Biotechnology Co., Ltd. The primer sequences: the primer for the heavy chain variable region: 5′-ACG ACG TTG TAA AAC GAC GGC-3′(21mer) and the primer for the light chain variable region: 5′-ACG ACG TTG TAA AAC GAC GGC-3′(21mer) and 5′-CAG GGG CCA GTG GAT AGA CAG ATG G-3′(21mer).

Gene sequencing results: The heavy chain variable region encoding gene sequence is 339 bp in length, and is as shown in SEQ ID NO: 1. It is deduced from the gene sequence obtained that the heavy chain variable region encoded by the gene sequence consists of 113 amino acids, and has a sequence as shown in SEQ ID NO: 3. The light chain variable region encoding gene sequence is 315 bp in length and is as shown in SEQ ID NO: 2. It is deduced from the gene sequence obtained that the light chain variable region encoded by the gene sequence consists of 105 amino acids, and has a sequence as shown in SEQ ID NO: 4.

5. Preparation, Purification, and Subtype and Characteristic Identification of Anti-Carbaryl Monoclonal Antibody

The hybridoma cell line Jnw1D2 producing anti-carbaryl monoclonal antibody was injected into BALB/c mice previously treated with incomplete Freund's adjuvant. The ascetic fluid was collected from the mice and the antibody was purified by caprylic acid-ammonium sulfate precipitation. The operation was specifically as follows. The ascetic fluid was filtered through double-layered filter paper, and centrifuged at 12000 r/min for over 15 minutes at 4° C. The supernatant was aspirated, and mixed with 4 times volume of an acetate buffer. n-caprylic acid was added with stirring in an amount of 30-35 μL per ml ascetic fluid, mixed for 30-60 min at room temperature and allowed to stand at 4° C. for more than 2 h. After centrifugation at 12000 r/min for over 30 minute at 4° C., the pellet was discarded, and the resulting supernatant was filtered through double-layered filter paper. A 0.1 mol/L phosphate buffer (pH 7.4) that was 1/10 the volume of the filtrate was added, and the pH of the mixture was adjusted to 7.4 with a 2 mol/L sodium hydroxide solution. Ammonium sulfate was slowly added in an ice bath until the final concentration of ammonium sulfate was 0.277 g/mL, and the mixture was allowed to stand at 4° C. for 2 h or more. Then, the mixture was centrifuged at 12,000 rpm for over 30 minutes at 4° C. The supernatant was discarded, and the resulting pellet was re-suspended in a 0.01 mol/L phosphate buffer (pH 7.4) that was 1/10 the volume of the original ascetic fluid, filled into a dialysis bag, and dialyzed for two days against 0.01 mol/L PBS and then against PB for two days. The protein solution was removed from the dialysis bag and centrifuged. The pellet was discarded, and the supernatant was collected, pre-frozen at −70° C. and then lyophilized in a lyophilizer. The lyophilized powder was collected, which was the purified anti-carbaryl monoclonal antibody.

The acetate buffer contained 0.29 g of sodium acetate, 0.141 mL of acetic acid and water q.s. to 100 mL. The 0.01 mol/L phosphate buffer contained 0.8 g of sodium chloride, 0.29 g of disodium hydrogen phosphate dodecahydrate, 0.02 g of potassium chloride, 0.02 g of potassium dihydrogen phosphate, and water q.s. to 100 mL. The 0.1 mol/L phosphate buffer contained 8 g of sodium chloride, 2.9 g of disodium hydrogen phosphate dodecahydrate, 0.2 g of potassium chloride, 0.2 g of potassium dihydrogen phosphate, and water q.s. to 100 mL.

The subtype of the anti-carbaryl monoclonal antibody secreted by the hybridoma cell line Jnw1D2 was identified as IgG2b by using a commercial subtype identification kit. The titer of the antibody was 1.6×10⁴ measured by enzyme-linked immunosorbent assay (ELISA). The 50% inhibitory concentration (IC₅₀) for carbaryl was 0.668 ng/kg, and there was no cross reaction with carbofuran, aldicarb, and methomyl, etc.

### EXAMPLE 1

### Preparation of Fluorescent Marker

Step 1: Preparation of Intermediate (1-naphthyloxy-4-nitrophenyl carbonate) for Synthesizing Carbaryl Hapten

In a 1 L four-necked flask equipped with an electric stirrer, a thermometer and a constant pressure dropping funnel, 240 mL of dichloromethane, 33 mL of triethylamine, and 31.7 g of naphthol were added at room temperature, stirred until they are dissolved, and then cooled to 0° C. in a low temperature reaction bath. 40.2 g of p-nitrophenyl chloroformate was dissolved in 60 mL of a methylene chloride solution, and slowly added dropwise to the above solution, during which a white smoke was produced and the color of the solution became darker with the dripping. After the addition was completed in 1 h, the reaction was incubated for 3 h, and the reaction was detected to be complete by TLC (developing agent: methylene chloride:petroleum ether=1:3). 360 mL of 3% hydrochloric acid was added and stirred for about 30 min. The organic phase was separated, combined, washed twice with 300 mL of water until neutral, dried over anhydrous sodium sulfate and concentrated under reduced pressure to about 60 mL. 180 mL of methyl tert-butyl ether was added and a solid was precipitated out upon cooling, which was suctioned to obtain a white solid that was the intermediate for synthesizing carbaryl hapten.

Step 2: Synthesis of Carbaryl Hapten CNH

In a 1 L four-necked flask equipped with an electric stirrer, a thermometer and a constant pressure dropping funnel, 360 mL of a saturated sodium bicarbonate solution and 9.6 g of 6-aminohexanoic acid were added at room temperature, stirred until the solid was dissolved, and then cooled to 0° C. in a low temperature reaction bath. 12 g of the intermediate (where the molar ratio of the intermediate to 6-amino hexanoic acid was 1: 2) was dissolved in 360 mL of tetrahydrofuran, and then slowly added dropwise to the above solution, during which the color of the solution became yellow, and a solid was gradually precipitated out. After the addition was completed in 1 h, the resultant system was stirred overnight at room temperature, and suctioned. The filtrate was adjusted to pH 4-5 with 3 mol/L hydrochloric acid, and extracted three times with 300 mL of ethyl acetate. The organic phases were combined and dried over anhydrous sodium sulfate. The solvent was removed under reduced pressure to give 10 g of a yellow oil, which was recrystallized in 10 mL of ethyl acetate and 30 mL of methyl tert-butyl ether to give a pale pink solid.

Step 3: Synthesis of Fluorescein Isothiocyanate Ethylenediamine (EDF)

200 mg (1.5 mmol) of ethylenediamine hydrochloride was dissolved in a mixed solution of 50 mL methanol and 0.5 mL triethylamine mixture, and the resulting solution was designated as solution A. 117 mg (0.3 mmol) FITC was dissolved in a mixed solution of 10 mL methanol and 100 μL triethylamine, and the resulting solution was designated as solution B. The solution B was added dropwise to the solution A within 30 min. The reaction was stirred for 2 h at room temperature in the dark, and allowed to stand overnight in the dark. The resulting orange precipitate was filtered with filter paper, washed with 10 mL of methanol and allowed to stand and air dry at room temperature in the dark, to obtain EDF. The synthesis of fluorescein BDF and HDF was similar.

Step 4: Synthesis of Fluorescent Marker

For example, CNH-EDF was synthesized as follows.

4 mg of DCC and 2 mg of NHS were weighed and added to 500 μL of DMF. After mixing well, 3 mg carbaryl hapten CNH was added, and reacted for 12 h at room temperature with agitation. Then 2 mg of EDF was added to the above activated CNH and the reaction was continued for 4 h at room temperature in the dark. 50 μL of the reaction solution was separated by thin layer chromatography (TLC) with the developing solvent chloroform/methanol (v:v, 4:1). The yellow band with R_{f}=0.9 was scraped from the silica gel plate, eluted with methanol, and detected for later use. MS for CNH-EDF: m/z 733.23 [M+H]⁺.

The reaction steps for other fluorescent markers, CNH-BDF and CNH-HDF, were similar to the labelling method for EDF, and the fluorescent markers were stored at 4° C.

### EXAMPLE 2

### Screening of Optimum Fluorescent Marker

Step 1: First, the working concentration of each fluorescent marker was set to a corresponding fluorescent marker concentration (5 nM) when the fluorescence intensity was 10 times of the background fluorescence intensity of the borate buffer solution. The antibody was diluted in the times of 125, 250, 500, 1000, 2000, 4000, 8000, 16000 and 32000 with a borate buffer solution to plot an antibody binding curve, so as to obtain the maximum change δmP in the signal intensity (δmP=mPₘₐₓ−mPₘᵢₙ). CNH-EDF had the largest change in signal intensity. The experimental results are shown in Table 2:

TABLE 2
Signal intensity of three fluorescent markers binding to antibody

| Fluorescent marker | δ mP |
|---|---|
| CNH-EDF | 231 |
| CNH-BDF | 124 |
| CNH-HDF | 102 |

Step 2: First, the working concentration of each fluorescent marker was set to a corresponding fluorescent marker concentration (5 nM) when the fluorescence intensity was 10 times of the background fluorescence intensity of the BB solution (borate buffer solution). With a corresponding antibody dilution factor when 70% of the anti-carbaryl monoclonal antibody binds to the fluorescent marker (1 μg/mL), the carbaryl detection standard curve of different markers was established and the IC₅₀ was calculated. The optimum fluorescent marker was decided based on the IC₅₀ value of each standard curve. The experimental results are shown in Table 3.

TABLE 3

| Fluorescent marker | IC₅₀ (ng/mL) |
|---|---|
| CNH-EDF | 88.5 |
| CNH-BDF | 150.5 |
| CNH-HDF | 170.8 |

It can be known from Table 3 that the optimum fluorescent marker is CNH-EDF.

### EXAMPLE 3

### Establishment of FPIA Method

Step 1: Competitive FPIA: formulation of borate buffer solution: 0.47 mg Na₂B4O₇, and 0.05 mg NaN₃ were weighed and dissolved in 0.5 mL of high purity water, and the pH value was 8.5.

9 concentration gradients of carbaryl standards of 100 μg/mL, 10 μg/mL, 1 μg/mL, 0.5 μg/mL, 0.1 μg/mL, 0.05 μg/mL, 0.01 μg/mL, 0.005 μg/mL and 0.001 μg/mL were formulated with 10% methanol in a borate buffer solution, and each 50 μL of the carbaryl standards, 500 μL fluorescent marker of working concentration (5 nM), and 500 μL anti-carbaryl monoclonal antibody of the working concentration (1 μg/mL) were added to a reaction tube, and incubated at room temperature in the dark for 10 min. Then the fluorescence polarization value was measured at an excitation wavelength of 485 nm and an emission wavelength of 530 nm, with a cutoff of 515 nm.

Step 2: Plotting of the standard curve: After the competitive reaction was finished, a standard curve of the determined fluorescence polarization values as longitudinal coordinates against the concentrations of the carbaryl standards as horizontal coordinates was fitted by a four-parameter model of Origin 9.0.

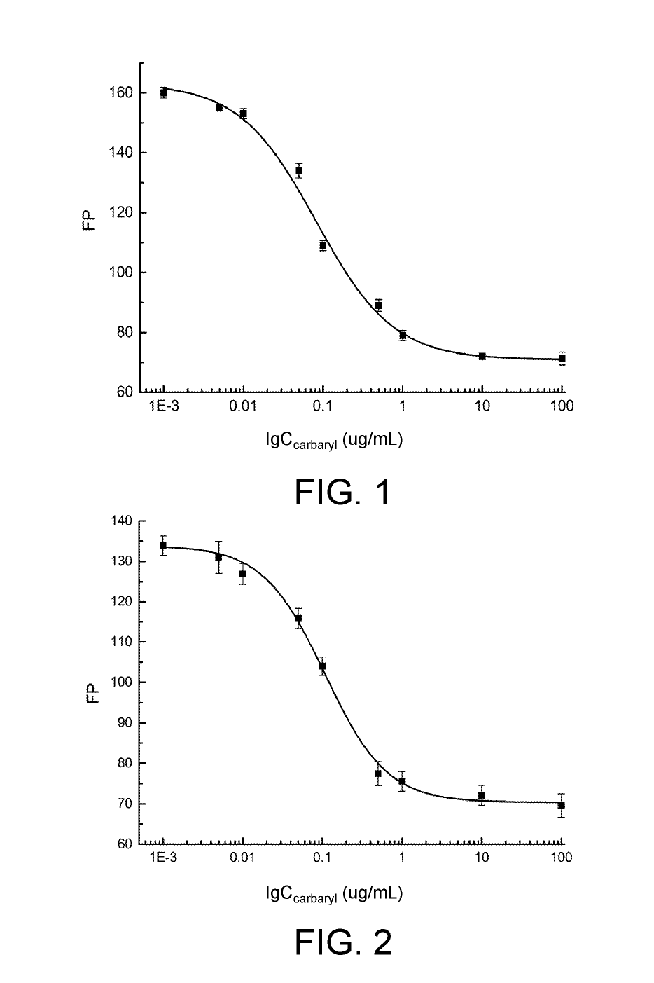

The fluorescence polarization standard curve of carbaryl in borate buffer solution is shown in FIG. 1.

The established standard curve has a sensitivity of 82.3 ng/mL and a detection range of 17.7-383.4 ng/mL.

### EXAMPLE 4

### Application Example—Sample Detection

Step 1: 20.0 g of the blank strawberry sample (which was determined to be carbaryl free by LC) was weighed and added to 20.0 mL acetonitrile, homogenized for 2 min at a high speed, filtered into a 50 mL centrifuge tube containing 4 g of sodium chloride, shaken vigorously for 3 min, and centrifuged at 5000 g for 2 min. 10 mL of the supernatant extract was aspirated to a beaker, heated in a water bath at 80° C., and evaporated to almost dryness while nitrogen was introduced into the beaker. 10 mL of methanol was added to dissolve the sample residue, to obtain a sample matrix solution. 9 concentration gradients of carbaryl standards of 100 μg/mL, 10 μg/mL, 1μg/mL, 0.5 μg/mL, 0.1 μg/mL, 0.05 μg/mL, 0.01 μg/mL, 0.005 μg/mL and 0.001 μg/mL were formulated with the sample matrix solution. Each 50 μL of the carbaryl standard solution in the sample matrix solution, 500 μL fluorescent marker solution and 500 μL anti-carbaryl monoclonal antibody solution were added and incubated at 25° C. for 5 min. Then FPIA detection was conducted. According to the correlation between the fluorescence polarization signal and the concentration of the carbaryl standard, a standard curve for carbaryl detection in the strawberry sample was obtained, as shown in FIG. 2. The detection sensitivity of carbaryl in the strawberry matrix is 108.6 μg/kg and the detection range is 32.4-363.6 μg/kg. The international standard stipulates that the maximum residue level of carbaryl in fruit is 1 mg/kg. Therefore, the method of the present invention is capable of well meet the requirement of detection sensitivity.

Step 2: Determination of recovery rate after addition: Carbaryl standard was added to the blank strawberry matrix to give a final concentration of 50 μg/kg, 100 μg/kg and 200 μg/kg, where each concentration was triplicated. The samples were processed and tested as described above, and the recovery rate was calculated according to a formula below.

Recovery rate (%)=(Measured/Added)×100%

The calculated recovery rate was used to evaluate the accuracy of the FPIA method for detecting carbaryl established in the present invention. The experimental results are shown in Table 4. The experimental results are shown in Table 4.

TABLE 4
Recovery rate after adding carbaryl to the strawberry matrix (n = 3)

| Added (μg/kg) | Recovered (μg/kg) | Recovery rate (%) | CV (%) |
|---|---|---|---|
| 50 | 49.2 | 98.4 | 3.9 |
| 100 | 105.6 | 105.6 | 5.6 |
| 200 | 180.5 | 90.2 | 6.7 |

As can be seen from Table 4, the average recovery rate of carbaryl in the strawberry matrix is in the range of 90.2 to 105.6% with a coefficient of variation (CV) of less than 6.7%. The results show that the FPIA method for detecting carbaryl established in the present invention can meet the requirement of carbaryl residue detection in strawberry; and the method of present invention is fast, efficient, and sensitive, and can be well used in the fast and highly sensitive detection of carbaryl by solving the disadvantages of complex operation and waste of time existing in the conventional immunoassay method.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A fluorescence polarization immunoassay (FPIA) method for detecting carbaryl, comprising the steps of mixing a sample to be tested, a fluorescent marker solution and an anti-carbaryl monoclonal antibody solution; incubating for competitive reaction; determining a fluorescence polarization value of the resulting system; and
calculating the concentration of carbaryl in the sample to be tested according to a standard curve between the known concentrations of carbaryl and the corresponding fluorescence polarization values.

2. The FPIA method for detecting carbaryl according to claim 1, wherein the standard curve between the known concentrations of carbaryl and the corresponding fluorescence polarization values is obtained by mixing a series of given concentrations of carbaryl standard solutions respectively with the fluorescent marker solution and the anti-carbaryl monoclonal antibody solution, incubating for competitive reaction, and determining the fluorescence polarization values of the resulting systems; and plotting a standard curve of the determined fluorescence polarization values as longitudinal coordinates against the concentrations of the series of given concentrations of carbaryl standard solutions as horizontal coordinates.

3. The FPIA method for detecting carbaryl according to claim 1, wherein the fluorescent marker is a conjugate of a carbaryl hapten 6-(1-naphthyloxyformamido)-hexanoic acid with fluorescein via an amide linkage.

4. The FPIA method for detecting carbaryl according to claim 3, wherein the fluorescein is any one selected from fluorescein isothiocyanate ethylenediamine, fluorescein isothiocyanate butanediamine, and fluorescein isothiocyanate hexamethylenediamine, and the fluorescent marker has a structure as shown in a table below: 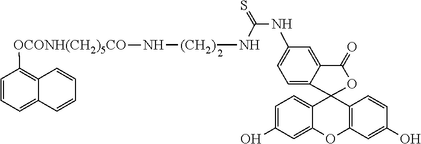
CNH-EDF 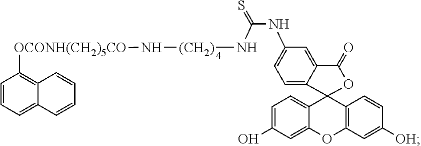
CNH-BDF
or 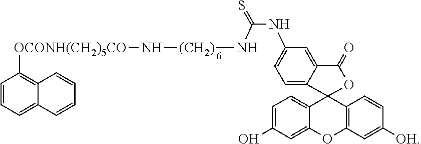
CNH-HDF

5. The FPIA method for detecting carbaryl according to claim 1, wherein the anti-carbaryl monoclonal antibody is secreted by the hybridoma cell line Jnw1D2 deposited in China Center for Type Culture Collection (CCTCC), Wuhan University, Wuhan, China on Mar. 29, 2016 with the accession number of CCTCC No. C201654.

6. The FPIA method for detecting carbaryl according to claim 1, wherein the solvent for formulating the fluorescent marker solution is a borate buffer solution; and
the working concentration of the fluorescent marker is a corresponding concentration at which the fluorescence intensity of the fluorescent marker is 10 times of the background value of the borate buffer solution, and is 5 nM; and
the working concentration of the anti-carbaryl monoclonal antibody is 1 μg/mL which corresponds to the antibody dilution factor when 70% of the anti-carbaryl monoclonal antibody binds to the fluorescent marker.

7. The FPIA method for detecting carbaryl according to claim 1, wherein the competitive reaction occurs at 20-25° C., and is continued for 5-10 min.

8. The FPIA method for detecting carbaryl according to claim 1, wherein the fluorescence polarization value is determined at an excitation wavelength of 485 nm and an emission wavelength of 530 nm.

9. The FPIA method for detecting carbaryl according to claim 1, wherein the sample to be tested is an agricultural product.

10. The FPIA method for detecting carbaryl according to claim 9, wherein the agricultural product is strawberry, wherein
a plurality of pretreatment procedures for strawberry samples before detection are as follow: the strawberry sample was mashed and homogenized with acetonitrile, then filtered into a centrifuge tube containing sodium chloride to collect the filtrate. After oscillating for a while, the sample was centrifuged to obtain the supernatant extract. Then the solvent of the supernatant was evaporated. The sample residue was dissolved in methanol to obtain the sample matrix solution, when the sample to be tested is strawberry, a series of given concentrations of carbaryl standard solutions are formulated with the sample matrix solution obtained by pre-treating a blank strawberry sample, and amenable to FPIA assay, to obtain a standard curve of the strawberry matrix.
